(19) United States
(12) Patent Application Publication
(10) Pub. No.: US 2020/0253516 A1
(43) Pub. Date: Aug. 13, 2020

(54) PULSE OXIMETER USING DISPOSABLE MULTI-MATERIAL STRETCH BANDAGE

(71) Applicant: KONINKLIJKE PHILIPS N.V., EINDHOVEN (NL)

(72) Inventors: John CRONIN, BONITA SPRINGS, FL (US); Michael D'ANDREA, BONITA SPRINGS, FL (US)

(21) Appl. No.: 15/776,201

(22) PCT Filed: Nov. 23, 2016
(86) PCT No.: PCT/EP2016/078519

§ 371 Date: May 15, 2018

Related U.S. Application Data
(60) Provisional application No. 62/259,062, filed on Nov. 24, 2015.

(30) Foreign Application Priority Data
Mar. 18, 2016 (EP) 16161070.4

(51) Int. Cl. A61B 5/1455; A61B 5/00

(52) U.S. Cl. CPC A61B 5/14552; A61B 5/6826; A61B 5/6829; A61B 2562/16; A61B 2560/0285; A61B 2503/04; A61B 5/683

(57) ABSTRACT

A customizable pulse oximetry assembly is disclosed. The assembly include a bandage with a first receptacle, a second receptacle, and a flexible connector connecting the first receptacle and the second receptacle. The assembly also includes a light emitter having a first probe housing for removably mounting the light emitter on the first receptacle and a light sensor comprising a second probe housing for removably mounting on the second receptacle. A cable connector for physically coupling the light emitter and the light sensor and for communicatively coupling the light emitter and the light sensor to a patient monitor is included as a part of the assembly. The flexible connector, the first probe housing, and the second probe housing may be adapted to adjust for a separation and an alignment between the light emitter and the light sensor according to a dimension of a patient body part.

### FIELD OF THE INVENTION

The present invention generally relates to pulse oximetry assemblies. More specifically, the present invention concerns a pulse oximetry assembly having a flexible connector and receptacles for mounting a light emitter and a light sensor.

### DESCRIPTION OF THE RELATED ART

Pulse oximetry is an effective and non-invasive method of monitoring and acquiring oxygen saturation (SpO₂) level and perfusion index of a patient. Pulse oximetry requires illumination of the patient blood-perfused tissue using a light source at one or more wavelengths. Spectroscopy is then employed to determine any changes in the oxygen saturation level, which can provide an indication of a patient condition. Because of the way pulse oximetry acquires light-based physiological data, it is critical to ensure the alignment of the light-emitting and sensing components to maximize the amount of desired detected signal and minimize detected signal contribution.

To align the light-emitting and sensing components, pulse oximeters typically use clip-on devices made of rigid materials. Although these afford secure placement on or attachment to a patient body part, clip-on devices cannot accommodate patient body parts with significantly varying sizes. Flexible oximeter devices offer an advantage over clip-on pulse oximeters in terms of adaptability for use with patient body parts of varying dimensions. The light-emitting and light-sensing components nevertheless remain difficult to align in flexible oximeter devices.

For example, U.S. patent publication number discloses a flexible oximetry assembly for mitigating undesired effects of patient motion on oximeter readings. The flexible oximeter includes an elastic sensor body with a stiffening member positioned between an emitter and a detector for stabilizing the distance between the emitter and the detector. The stiffening member is adapted to bend to establish a fixed distance between the emitter and detector.

As a further example, U.S. Pat. No. 8,706,179 discloses a disposable bandage apparatus having a probe emitter receptacle and a probe detector receptacle for removably mounting an emitter and a detector, respectively. The apparatus also includes a biasing member for ensuring the alignment between the emitter and the detector.

There remains a need in the art for a pulse oximeter assembly capable of being adjusted to ensure a separation and an alignment between the light emitter and the light sensor while nevertheless addressing the dimensions of a patient body part.

### SUMMARY OF THE PRESENTLY CLAIMED INVENTION

A first embodiment of the present invention concerns a customizable pulse oximetry assembly having a bandage with a first receptacle, a second receptacle, and a flexible connector connecting the first receptacle and the second receptacle. The assembly includes a light emitter having a first probe housing for removably mounting the light emitter on the first receptacle and a light sensor comprising a second probe housing for removably mounting on the second receptacle. The assembly further includes a cable connector for physically coupling the light emitter and the light sensor and for communicatively coupling the light emitter and the light sensor to a patient monitor. The flexible connector, the first probe housing, and the second probe housing are adapted for adjusting a separation and an alignment between the light emitter and the light sensor according to a dimension of a patient body part.

A second embodiment of the present invention includes a method for attaching a light emitter having a first probe housing and a light sensor having a second probe housing to a bandage. The bandage includes a first receptacle for the first probe housing, a second receptacle for the second probe housing, and a flexible connector connecting the first receptacle to a second receptacle. The bandage is extended via the flexible connector to accommodate a dimension of a patient body part. The bandage is attached to a patient body part. The bandage may be adjusted such that the light emitter and the light sensor are aligned. Light sensor data is then acquired using the light emitter and the light sensor. Data may then be transmitted to a patient monitor via a cable connector.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention.

FIG. 1A illustrates a customizable pulse oximetry assembly.

FIG. 1B illustrates the pulse oximetry assembly in a contracted state.

FIG. 1C illustrates the pulse oximetry assembly in a stretched state to accommodate a physical dimension of a patient body part.

FIG. 2 illustrates a method for measuring pulse oximeter data using the pulse oximetry assembly of the present invention.

FIG. 3A illustrates the pulse oximetry assembly attached to a small finger.

FIG. 3B illustrates the pulse oximetry assembly attached to a larger finger.

FIG. 3C illustrates the pulse oximetry assembly 100 attached to an infant foot.

### DETAILED DESCRIPTION OF THE EMBODIMENTS

The following are definitions of terms as used in the various embodiments of the present invention:

The term “receptacle” as used herein refers to a structural component for mounting a light emitter or a light sensor. The receptacle may comprise rigid components (e.g., a metal, glass, or rigid polymer), flexible components (e.g., polymer or rubber), or any combination thereof.

The term “flexible connector” as used herein refers to a physical connector for connecting two receptacles and adjusting the separation and orientation between the two receptacles. The flexible connector can be a wire, mesh, polymer, a series of pivotally connected segments, or any combination thereof, capable of being extended or contracted in length.

The term “bandage” as used herein refers to an assembly comprising two receptacles connected by the flexible connector.

The term “probe housing” as used herein refers to an enclosure for enclosing a light emitter or a light sensor. The probe housing can be flexible and further usable for mounting the light emitter or the light sensor on the receptacles. The probe housing may comprise a material such as a polymeric outer material and a flexible inner material comprising a metal or a polymer configured as wires, mesh, plate, or any configuration adapted to be flexible and structurally stable upon bending.

The term “cable connector” as used herein refers to a cable for physically connecting the light emitter and the light sensor and for communicatively coupling the light emitter and the light sensor to an external device, such as a patient monitor. The cable connector can be a Y-cable wherein one end connects to an external system and the other end with the Y-branch connects to the light emitter and the light sensor. The cable connector can be a USB cable capable of transmitting data packets and power from one end of the cable to another. The cable connector can also be a fiber optic cable.

With the foregoing definitions in mind, pulse oximetry is a method for measuring the oxygen saturation of patient blood. Blood oxygen saturation is a measure of the amount of oxygen carried by hemoglobin in the blood stream. Saturation is usually expressed as a percentage rather than an absolute reading.

For example, blood oxygen saturation levels measured immediately after birth can provide a good indicator of a general state of health. Levels below 75% could indicate that the newborn infant may be suffering from some abnormality. To determine a patient condition, the blood oxygen saturation should be expressed as a percentage of the total hemoglobin that is saturated with oxygen. Acceptable normal ranges for healthy patients range from 95 to 99 percent.

A pulse oximeter in accordance with the present invention includes an electronic processor and two or more small light-emitting diodes (LEDs) or a series of LEDs facing a photodiode at a translucent portion of a patient body. This is typically a fingertip or an earlobe. One of the LEDs emits light in the red portion of the visible portion of the electromagnetic spectrum (red LED) while the other LED emits in the infrared portion. The amount of light absorbed at these two wavelengths differs significantly between oxygen-rich blood and blood deficient in oxygen as oxygenated hemoglobin absorbs more infrared light and allows more red light to pass through. Deoxygenated hemoglobin allows more infrared light to pass through and absorbs more red light. Oxy-hemoglobin and its deoxygenated form have significantly different absorption pattern.

In operation, the LEDs of the present invention alternately turn on and off and then both off for a predetermined period of time. This alternation and termination allows the light sensor such as a photodiode to separately respond to the red and infrared light and to correct for the light detected due to ambient light, which is measured when both LEDs are off and used as baseline or reference signal. The amount of light that is transmitted (that is, not absorbed) is measured and separate normalized signals are produced for each wavelength.

These signals vary with time because the amount of arterial blood that is present increases with each heartbeat. By subtracting the minimum transmitted light from the peak transmitted light in each wavelength, the effects of other tissues and materials (e.g., venous blood, skin, bone, muscle, fat, including nail polish) can be corrected in that they normally absorb a constant amount of light over a period of time. The ratio of the measured red light to the measured infrared light is calculated by the processor. This ratio, which represents the ratio of oxygenated hemoglobin to deoxygenated hemoglobin, is then converted to an oxygen saturation level reading by the processor.

The light emitter of the presently disclosed invention can be a light emitting diode, photodiode, phototransistor, photo emitter, or any other type of light emitting component capable of emitting light at one or more wavelengths and varying the intensity and duration of the emitted light. The light sensor may be a photodetector, photoelectric receiver, photodiode, or any other sensor capable of detecting light with one or more wavelengths or intensity. The patient monitor transmits operational data to and receives operational data from the light emitter and the light sensor via the cable connector. Operational data may include wavelength, light intensity, duration, and sampling rate.

The light emitter and the light sensor each include a probe housing for mounting the light emitter or light sensor on their respective receptacles. The probe housings are configurable to establish sufficient contact with the patient body part. The probe housings can also be physically manipulated to permit alignment of the light emitter and light sensor and to otherwise adapt to the shape of the surface of the patient body part to which they are attached. One form of physical manipulation includes bending the probe housing to conform to the shape of the surface of the patient body part. For example, a patient body part may have a relatively flat surface such that the probe housing is configured to also be also flat while another patient body part may be curved such that the probe housing is bent to conform to the curved surface.

The probe housing preferably incorporates one or more perforations to allow the transmission of light from the light emitter to the light sensor with minimal intensity loss. Alternatively, the probe housing may fully enclose the light emitter or the light sensor. In such an embodiment, the housing is preferably optically transparent at the wavelengths at which the light emitter and the light sensor operate.

The patient monitor of the presently disclosed invention includes a processor, display, user interface, and memory. The patient monitor further includes a communication module for transmitting or receiving data with an external system wirelessly or via wired connections. The patient monitor may also include a cable connector port for connecting a cable connector to the patient monitor.

The patient monitor is capable of performing functions such as transmitting data to and receiving data from the light emitter and light sensor via a cable connector. The monitor may also receive input from a user via the user interface as well as process data, store data, and display data. The monitor may also communicate with an external system, such as a cloud server, a hospital server, or a device such as mobile device, or a desktop computer.

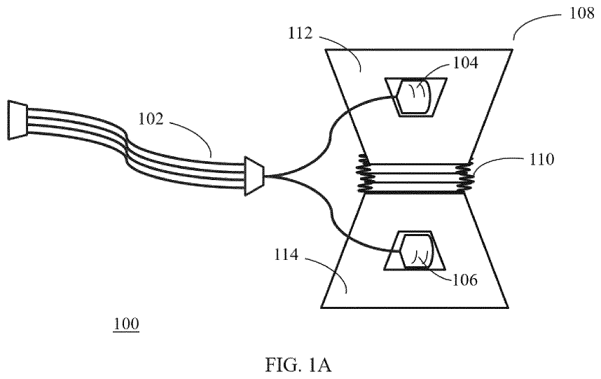

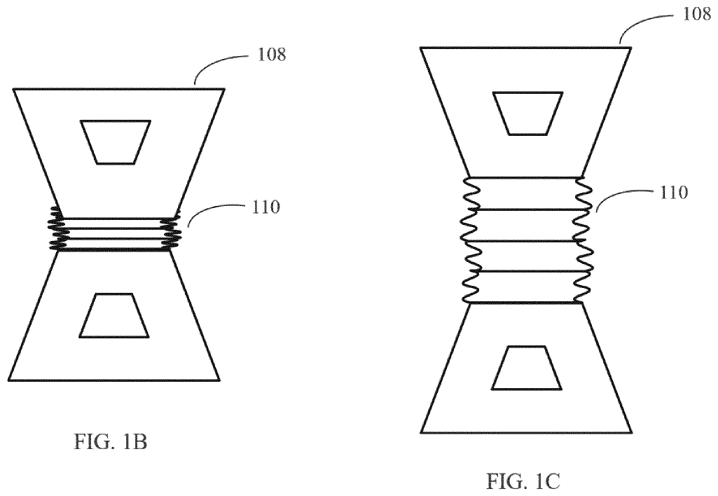

FIG. 1A illustrates a customizable pulse oximetry assembly 100. As shown in FIG. 1A, the assembly includes a cable connector 102, a light emitter 104, a light sensor 106, and a bandage 108 having a flexible connector 110 connected to receptacles 112 and 114. The light emitter 104 and light sensor 106 of FIG. 1A are respectively mounted on receptacles 112 and 114. The flexible connector 110 can be stretched, contracted, or distorted to accommodate the dimensions of a patient body part. FIG. 1B illustrates the pulse oximetry assembly 100 in a contracted state. FIG. 1C illustrates the pulse oximetry assembly 100 in a stretched state to accommodate a physical dimension of a patient body part.

Bandage 108 includes two receptacles 112 and 114 and a flexible connector 110 connecting the two receptacles 112 and 114. The receptacles 112 and 114 are usable for mounting the light emitter 104 and the light sensor 106. The flexible connector 110 allows for the adjustment of the separation and alignment of the light emitter 104 and the light sensor 106.

The receptacles 112 and 114 further include attachments for fastening the light emitter 104 and the light sensor 106 to the receptacles 112 and 114. A receptacle may incorporates any one or more of the following attachment means: Velcro, magnet, fastener, screw, clamp, clip, hook, pocket, strap, suction cup, pin, holes, and snaps. Other means for effectuating an attachment may also be used.

In one embodiment, the aforementioned attachments are positioned on the underside of the receptacle. As a result, the light emitter and the light sensor are directly in contact with the patient body part. In another embodiment, the attachments are positioned on the external side of the receptacle. As a result, the light emitter and the light sensor are mounted on the external sides of the receptacles by way of said attachments. In an embodiment where the receptacles are opaque and the light emitter and the light sensor are mounted on the external sides of the opaque receptacles, a window is built through the receptacles to allow transmission of light from the light emitter through the patient body part and from the patient body part to the light sensor.

The flexible connector is preferably made of a material such that in a stretched state as illustrated in FIG. 1C, the connector does not snap back into the original configuration as illustrated in FIG. 1B. The flexible connector might include metallic wires or a wire mesh that can be bent to assume any variety of configurations. The physical manipulations of the flexible connectors and probe housing such as bending, stretching, twisting, contraction, extension, or any combination thereof is reversible in a preferred embodiment.

The flexible connector may include a lightweight casing or sheath to protect the flexible connector. The flexible connector is preferably made of one or more materials that are both sturdy and nontoxic. For example, the flexible connector may include metallic wires wrapped by a rubber casing. Alternatively, the flexible connector includes a wire mesh enclosed in a plastic sheath. The choice of casing may be based on aesthetics or form factor. A medical facility or institution may design the casing to include labels or other useful information such as the name of manufacturer, hospital, or date of manufacture of the pulse oximetry assembly.

The flexible connector might also include a plurality of non-flexible sub-elements connected by hinges or similar connectors. As a result, the plurality of non-flexible sub-elements are collectively flexible as might occur in the case of a series of rigid segments pivotally interconnected in a serial manner. The series of rigid segments may be bent into a horseshoe shape with a variable curvature. The pivots for the individual sub-elements might further provide a certain amount of rigidity to maintain a bent configuration set by a medical personnel. As a result, the pivots afford sufficient rigidity to the interconnected plurality of sub-elements to allow them to retain their overall shape regardless of the physical distortions or manipulations at issue. The number of rigid segments used for the flexible connector could be varied depending on the one or more dimensions of the patient body part.

The flexible connector, in an embodiment, may have corrugations such that extending or contracting the flexible connector causes the corrugated flexible connector to contract, expand, unfold, and fold. As a result, at least some of the ridges and grooves of the corrugations overlap to more or less match the size of the patient body part. At least some portions of the corrugations may have Velcro-type hooks and loops to prevent the folded corrugated flexible connector from unintentionally unfolding.

The flexible connector of the bandage and the two receptacles may be integrated such that the flexible connector is not detachable from the two receptacles. The flexible connector and the receptacles may alternatively be detachable from each other. A detachable or replaceable flexible connector may permit a higher degree of customizability as a user could then replace the flexible connector or the receptacles if necessary.

For example, a user may replace a short flexible connector with a longer flexible connector or replace a long flexible connector with a shorter flexible connector. The user may also replace a worn-out or contaminated flexible connector with a new or sterilized flexible connector. Conversely, the user may replace a worn-out or contaminated receptacle with a new or sterilized flexible connector.

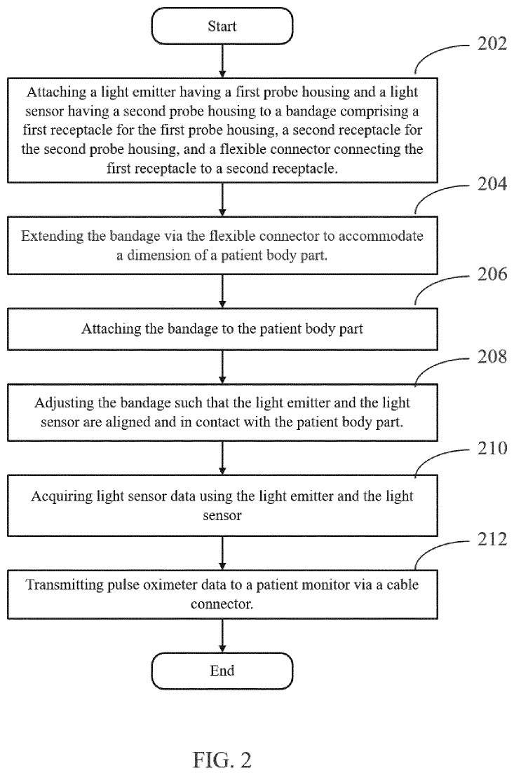

FIG. 2 illustrates a method for measuring pulse oximeter data using the pulse oximetry assembly of the present invention. A light emitter and a light sensor are initially attached to a bandage comprising a flexible connector (step 202). To conform to the shape and size of a patient body part, the assembly is extended via the flexible connector (step 204) and then attached to a patient body part to be used for the pulse oximetry measurement (step 206). The assembly is adjusted such that the attachment is stable and the light emitter and light sensor are aligned and in contact with the patient body part (step 208). The pulse oximetry data acquisition is then initiated by activating the light emitter and light sensor to acquire light sensor data (step 210). Afterwards, the acquired light sensor data are transmitted to a patient monitor via a cable connector for storage or further processing (step 212).

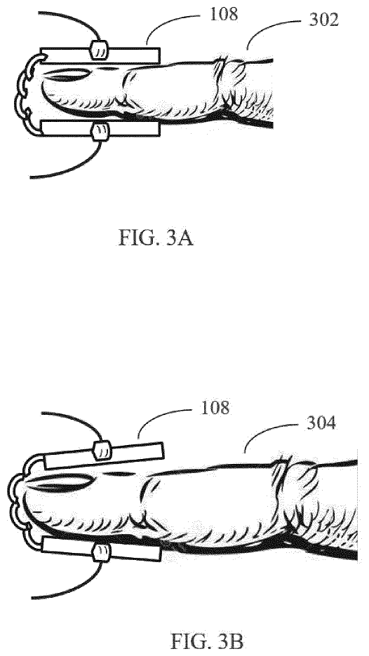

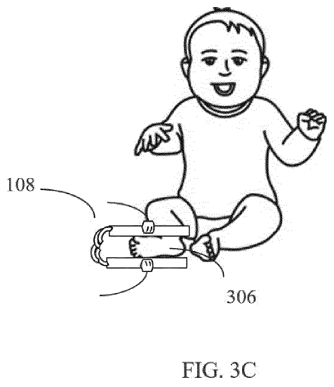

FIGS. 3A-3C illustrate various embodiments of the customizable pulse oximetry assembly as used on various patient body parts. FIG. 3A illustrates the pulse oximetry assembly 100 attached to a small finger 302. FIG. 3B illustrates the pulse oximetry assembly 100 attached to a larger finger 304. FIG. 3C illustrates the pulse oximetry assembly 100 attached to an infant foot 306. The pulse oximetry assembly of the present invention may also be used in other patient body parts, such as the ear, forehead, nose, and toe.

In an exemplary use case involving the present invention, three patients are admitted to an emergency room. The first patient is a 10 year-old child, the second an adult male, and the third is an infant. The medical personnel attends to the first patient and prepares a pulse oximeter assembly by first selecting a bandage and mounting the light emitter and light sensor on the bandage. The bandage is then attached to the finger of the child; the bandage is adjusted to conform to the shape and size of the finger of the child. The medical personnel then measures the blood oxygen saturation level of the child.

After the measurement, the medical personnel then attends to the second patient. The medical personnel uses the same pulse oximetry assembly for the adult patient. The pulse oximetry assembly is first wiped with a disinfectant, then the flexible connector is stretched to accommodate the second patient body part—such as a finger that would be larger than that of the first patient—after which the pulse oximeter data is measured.

The medical personnel then takes a pulse oximetry measurement of the infant patient. The medical personnel decides to use the same light emitter and light sensor after disinfecting them but uses a new bandage for the infant to minimize infections. The medical personnel then stretches the flexible connector so it fits the foot of the infant then acquires the infant pulse oximetry data.

After acquiring each pulse oximeter data, the medical personnel transfers the acquired data to the emergency room desktop computer for displaying and processing the acquired data. The medical personnel views the various data on the desktop computer, analyzes said data, arrive at a diagnosis, and enters findings and comments into the respective patient records. The used pulse oximetry assembly components may either be disposed or sterilized for reuse on another patient.

The pulse oximetry assembly might include flexible electronic elements, such as flexible displays, flexible power sources, flexible light sources, and flexible sensors. In one embodiment, the light sensor and the light emitter are both flexible to allow easier adjustments when fitting the pulse oximetry assembly to the patient body part. In another embodiment, the receptacles may include a flexible display for providing visual indication such as a flexible OLED display to indicate the degree of alignment for the light emitter and light sensor. The light emitter and the light sensor may also be powered by a flexible battery embedded in the bandage's receptacles.

The present invention is not intended to be restricted to the several exemplary embodiments of the invention described above. Other variations that may be envisioned by those skilled in the art are intended to fall within the disclosure.

1. A customizable pulse oximetry assembly, the assembly comprising:
a bandage with a first receptacle and a second receptacle;
a flexible connector connecting the first receptacle and the second receptacle;
a light emitter including a first probe housing that removably mounts the light emitter on the first receptacle;
a light sensor including a second probe housing that removably mounts on the second receptacle; and
a cable connector that physically couples the light emitter and the light sensor and further communicatively couples the light emitter and the light sensor to a patient monitor, wherein the flexible connector, the first probe housing, and the second probe housing may be adjusted relative to a separation and an alignment between the light emitter and the light sensor resulting from a dimension of a patient body part;
wherein the first receptacle, the second receptacle, and the flexible connector are individually detachable.

2. The assembly of claim 1, wherein the light emitter alternates operation for a predetermined period of time.

3. The assembly of claim 1, wherein the light sensor responds to both red and infrared light.

4. The assembly of claim 1, wherein the light sensor corrects for ambient light.

5. The assembly of claim 1, wherein the light emitter is selected from the group including an LED, a photodiode, a phototransistor, and a photo emitter.

6. The assembly of claim 1, wherein the light emitter operates on one or more wavelengths.

7. The assembly of claim 1, wherein the probe housing is flexible.

8. The assembly of claim 1, wherein the probe housing includes one or more perforations that allow for the transfer of light from the emitter to the sensor.

9. The assembly of claim 8, wherein the probe housing is optically transparent at at least one wavelength, the at least one wavelength being the wavelength on which the light emitter operates.

10. The assembly of claim 1, wherein at least one of the receptacles includes one or more attachments, the attachment being selected from the group including Velcro, a magnet, a fastener, a screw, a clamp, a clip, a hook, a pocket, a strap, a suction cup, a pig, a hole, or a snap.

11. The assembly of claim 10, wherein the attachment is positioned under the underside of the receptacle thereby allowing for direct contact of the light emitter and the light sensor with the patient body part.

12. The assembly of claim 10, wherein the attachment is positioned on the external side of the receptacle thereby allowing for the light emitter and the light sensor to be mounted externally.

13. The assembly of claim 1, wherein the receptacles are opaque and include a window to allow transmission of light from the light emitter through the patient body part and from the patient body part to the light sensor.

14. The assembly of claim 1, wherein the flexible connector includes a series of hinges.

15. The assembly of claim 14, wherein the hinges are pivotally and serially interconnected thereby allowing for manipulation into a horseshoe configuration.
